(19) United States
(12) Patent Application Publication
(10) Pub. No.: US 2010/0152597 A1
(43) Pub. Date: Jun. 17, 2010

(54) IMPLANTABLE TELEMETRIC DEVICE FOR HEART MONITORING

(76) Inventors: Gianni PLICCHI, Bologna (IT); Emanuela Marcelli, Bologna (IT); Laura Cercenelli, Castelbellino (IT)

Correspondence Address: STITES & HARBISON PLLC, 1199 NORTH FAIRFAX STREET, SUITE 900, ALEXANDRIA, VA, 22314, US

(21) Appl. No.: 12/632,973

(22) Filed: Dec. 8, 2009

(30) Foreign Application Priority Data
Dec. 16, 2008 (IT) BO2008A000749

(51) Int. Cl. A61B 5/0402; A61B 5/02

(52) U.S. Cl. 600/509; 600/481

(57) ABSTRACT

What is described is an implantable telemetric device for measuring the electromechanical parameters of the heart, characterized in that it comprises a single sensor (5) and corresponding processing means for detecting data relating to both the rotation of the heart and the mechanical vibrations which correspond to the first heart sound (FHS) and the second heart sound (SHS), and in that it comprises means which use these data for diagnostic and/or therapeutic purposes. Said sensor (5) is of the type which can generate a broadband electrical signal proportional to the rotation rate (RotR) of the heart and proportional to said mechanical vibrations corresponding to the heart sounds. Means are provided for processing this RotR signal, for deriving therefrom the functions relating to the rotation of the heart (∫RotRdt) and to mechanical vibrations (d(RotR)/dt) corresponding to the said heart sounds (FHS, SHS) and for detecting data derived from combination of the said functions (e.g. data relating to any time interval (DI) that may be present between the peak of said heart rotation signal (∫RotRdt) and the second heart sound (SHS)) and data derived from basic signal processing operations (i.e. sum, subtraction, multiplication, division, mean, calculation of the area underneath curves) applied to all said functions, and means are provided for using all said data for diagnostic and/or therapeutic and/or monitoring purposes.

The invention relates to a cardiac device which is associated with a catheter, which can be implanted endocardially or epicardially, and which is provided with means for detecting electromechanical parameters of the heart for diagnostic and/or therapeutic purposes, for example for monitoring the heart function in order to detect the onset of any alarm conditions which require the use of therapeutic protocols of the pharmacological type, or for controlling implantable therapeutic devices such as pacemakers, defibrillators or other devices.

Italian patent application BO2005A000165 filed on 17 Mar. 2005 and granted on 3 Jul. 2009 under No. 1 362 862 describes an implantable cardiac device which is essentially provided with a sensor for detecting the rotation of the heart and for generating an electrical signal representing this rotation, and is provided with means which process this signal and compare it with basal levels, and which are designed to interact with any implantable or external device for diagnostic and/or therapeutic and/or monitoring purposes. The sensor which detects the rotation of the heart is mounted on the distal end of a catheter which is implanted in the epicardium or in the endocardium or intramurally. The sensor may be composed of a small vibrating gyroscope which detects with high precision the Coriolis force due to the rotation rate of the heart and which produces a signal corresponding to this rotation rate.

U.S. Pat. No. 7,445,605 describes a device for monitoring cardiac function in humans, provided with at least one motion sensor for detecting the movement of the heart, which, among other reported possibilities, can be composed of a rotation sensor and which comprises analytical means connected to said sensor for operation, these means receiving the signal generated by this component in order to process it and compare it with a basal level of the heart movement, this level being for example that of the normal movement of the healthy heart, and being introduced into the system as the known reference and threshold level.

There is no specific reference in the prior art to the use of specific signals which are correlated with the physiological function of the patient to be associated with the heart movement signal, except in relation to the intracardiac electrocardiogram, or, in an indirect way, in U.S. Pat. No. 7,445,605, the mention of the possibility of combining the motion sensor with an implantable device such as a pacemaker and/or defibrillator, in which the parameters usually detected by said implantable devices are, in addition to the endocavitary electrocardiogram, respiratory parameters deduced from the measurement of endocavitary impedance. However, none of these signals mentioned in the prior art, nor the intracardiac electrocardiogram, nor the respiration parameters which may be derived from an impedance measurement can provide information on the actual mechanical phases of heart function, particularly the actual mechanical phases of the start and end of systole and diastole, which can be ascertained only by methods which enable the closing phases of the mitral and aortic valves to be detected, or by detecting the heart sounds, namely the first heart sound (FHS) and the second heart sound (SHS), these being detected by means of an implantable device. At present, these parameters can be derived from an echocardiogram or from external phonocardiography, in other words by methods not compatible with an autonomous implanted device, or can be derived from microaccelerometers positioned directly in the tip of an implantable electrode for cardiac stimulation, as described in U.S. Pat. No. 5,609,612, or positioned in a subcutaneous casing similar to those used for implantable cardiac electrostimulators, as described in U.S. Pat. No. 6,869,404B2, to which reference will mainly be made. In these prior patents, the said signals relating to the heart sounds are correlated and processed as a function of the electrical signals relating to the electrocardiogram, with which they must be suitably synchronized. The detection of the endocavitary electrogram, or the detection of the subcutaneous or surface ECG, would not be sufficient to identify precisely the start and end of the cardiac phases of systole and diastole, since the temporal correspondence between the electrical phases (detectable from the ECG) and the mechanical phases (systole and diastole) is not always identical and changes as a function of pathologies, the development of these, and contingent working conditions of the heart. For example, the time elapsing between the peak of the R wave and the start of mechanical systole varies greatly between a patient with a narrow QRS wave and a patient with a left bundle branch block. In the case of a left bundle branch block, there is a pathological delay between the QRS and the start of the systolic ejection phase, as well as an increase in the isovolumic contraction phase which precedes systole.

It is well known that in healthy subjects the cardiac rotation follows a specific pattern within the cardiac cycle in order to be effective: during isovolumic contraction all short-axis levels from base to apex rotate counterclockwise, when viewed from the apex, while, during ejection, the apex continues counterclockwise while the base reverses direction; in diastole, during isovolumic relaxation, the shearing forces built up during systole by cardiac torsion result in rapid untwisting which is thought to contribute to diastolic suction (see “J Biomech 1996; 29(6): 745-52”, “Am J Physiol Heart Circ Physiol 2000; 278(4): H1117-23”, “Ann Biomed Eng 1986; 14(6): 547-62”). Alterations in the normal pattern or magnitude of the cardiac rotation have been associated with cardiovascular diseases, such as chronic heart failure (see “Eur J of Heart Fail 2004; 6:715-722”), tachycardia-induced dilated cardiomyopathy (see “J Thorac Cardiovasc Surg 2002; 124:43-9”), dilated cardiomyopathy (see “J Thorac Cardiovasc Surg 2003; 126:48-55”), hypertrophic cardiomiopathy (see “Circulation 1992; 86(6):1919-28”), myocardial infarction (see “Coron Artery Dis 2000; 11(3):261-267”) and aortic stenosis (see “Eur Heart J 2000; 21:582-589”).

Thus, the analysis of the cardiac rotation signal with respect to systolic and diastolic phases is fundamental for the monitoring of cardiac function in patients with various degrees of heart failure (HF) or in non HF cardiopathic patients, who require, for example, monitoring of the effects of ischemia.

As for example, in cardiac insufficiency, there are often delay and/or dyssynchrony phenomena which are non-uniform mechanical movements of regions of the myocardium in the systolic and diastolic phases, which considerably reduce the overall mechanical efficiency of the heart, thus exacerbating and aggravating other cardiac insufficiency conditions. An important element of dyssynchrony has been identified in the time interval between the closing of the aortic valve (corresponding to the SHS) and the peak of the heart rotation signal at the end of systole (see “Am J Cardiol 2008; 101:1163-1169”). If this dyssynchrony time interval can be determined, it will be possible to achieve suitable adaptation of both the pharmacological treatment and the instrumental treatment in the form of the implantation of a so-called “biventricular” cardiac electrostimulator, in which the electrostimulation parameters relating to the right and left ventricles have the function of resynchronizing the heart mechanics and can be optimized if there is a knowledge of this dyssynchrony interval, which must be minimized.

The invention is intended to overcome these and other drawbacks of the known art with an apparatus as described in the appended Claim 1 and subsequent dependent claims, based on the following idea for a solution. Use is made of a sensor, preferably a single sensor, capable of providing measurements of both the rotation rate of the heart and the mechanical vibrations corresponding to the heart sounds (FHS, SHS), in such a way as to provide, by combination of these two measurements, a temporal characterization of the cardiac rotation signal with respect to the systolic and diastolic phase, in order to respond to problems of diagnosis and treatment which cannot be identified and resolved by the presence of a sensor which can only provide information on cardiac rotation.

The invention proposes the use of a gyroscopic sensor which can supply a broadband signal (0-1000 Hz) including both a measurement of the rotation rate of the heart, described in the literature as being in the 0-10 Hz band, and the detection of the mechanical vibrations corresponding to the heart sounds, which are described in the literature as being in the 15-200 Hz band. This broadband signal is processed by means of an integration operation, to supply a heart rotation angle signal, and is also processed by a derivation operation to find the FHS and SHS signals.

The association of the said signals, achieved by processing the broadband signal generated by the single rotation rate sensor, can be used for the accurate temporal characterization of the heart rotation signal with respect to the cardiac systolic and diastolic phases. It should be made clear that the identification of the mechanical systolic and diastolic phases, as achieved according to the present invention by the detection of the heart sounds (FHS, SHS), cannot be achieved in patients with suspected or evident cardiopathy solely by measuring the endocavitary electrocardiogram or the ECG (whether subcutaneous or surface), since the temporal correspondence between the start and the end of the mechanical systolic phase and the QRS and T waves changes as a function of numerous variables such as the pathologies, the development of the latter, the contractility of the myocardium, the heart rate, and the pre-loading and post-loading conditions. Especially in cardiopathic patients who require accurate measurement for the choice of appropriate treatment, the mechanical systolic and diastolic phases cannot be identified precisely by means of the ECG, because of the pathological electromechanical delays typically found in these patients. It should also be pointed out that the monitoring of the increase or decrease of these electromechanical delays and dyssynchronies provides essential information for the treating doctor or the consultant.

Further characteristics of the invention, and the advantages resulting therefrom, will be made clearer by the following description of a preferred embodiment of the invention, illustrated purely by way of non-limiting example in the figures on the four attached sheets of drawing, in which:

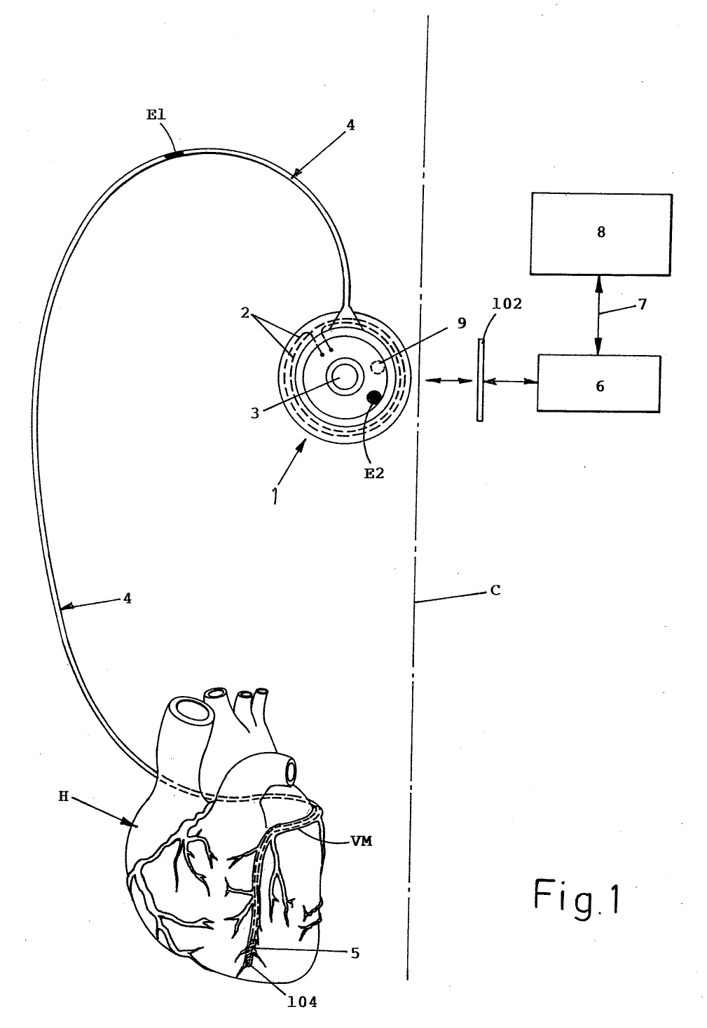

FIG. 1 is a schematic illustration of the device according to the invention, also shown in relation to external control and programming equipment;

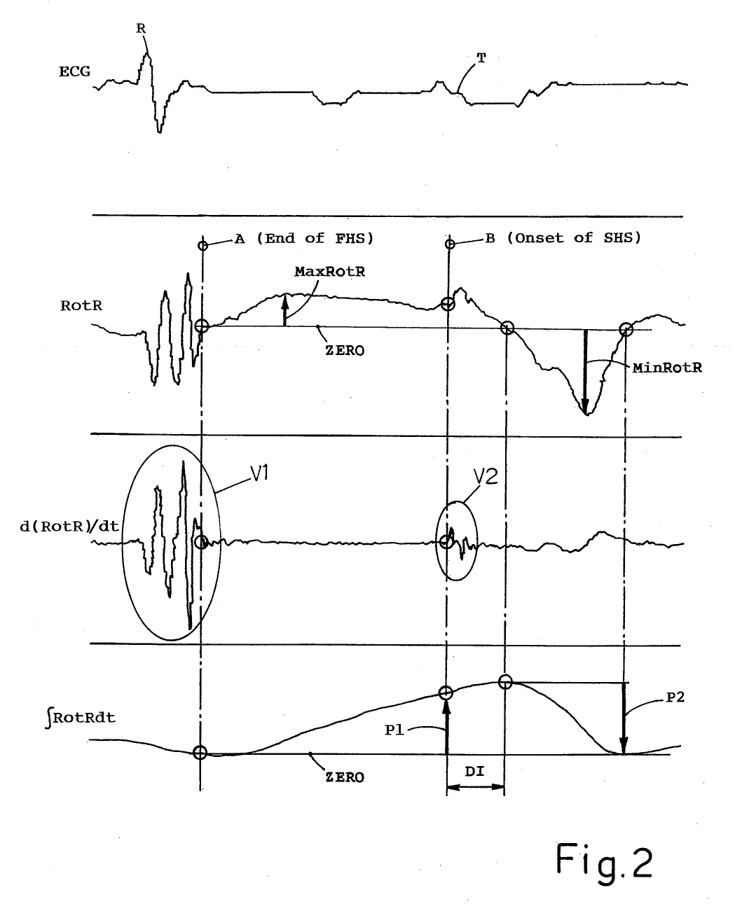

FIG. 2 shows a plurality of analogue signals obtained during experiments conducted on experimental animals to demonstrate the way in which the broadband “Rotation Rate” (RotR) signal generated by the sensor used in the device according to the invention is processed by the device to produce, by an operation of derivation, the signal for the mechanical vibrations corresponding to the heart sounds [d(RotR)/dt] and to find, by an operation of integration, the heart rotation signal (∫RotRdt) and an example of combination of the said processed signals to identify correctly the peaks of rotation rate during the ejection phase (MaxRotR) and the relaxation/refilling phase (MinRotR) of the heart chamber, and any dyssynchrony intervals (DI) corresponding to the time interval between the peak of said rotation signal (∫RotRdt) and the second heart sound (SHS);

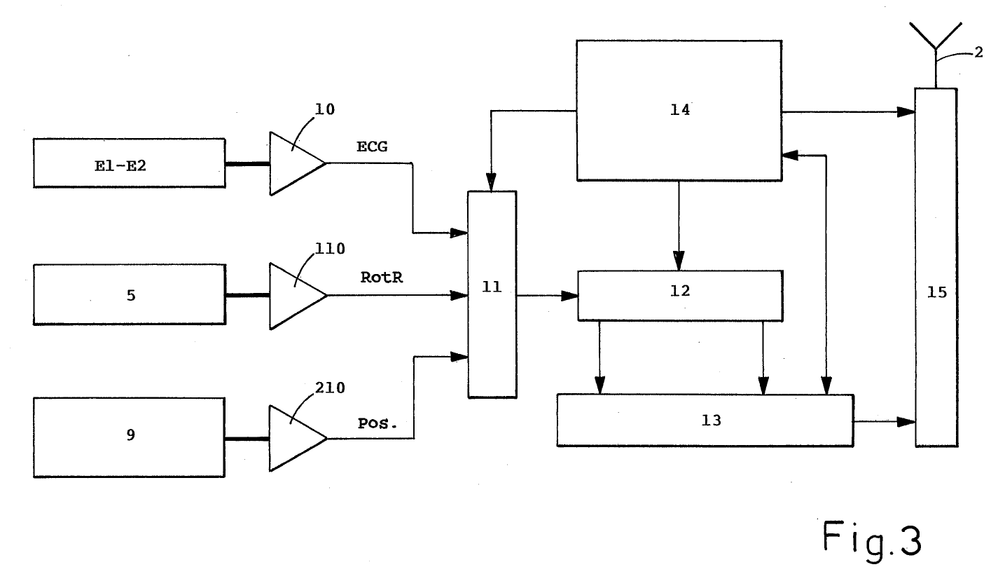

FIG. 3 is a block diagram of the implantable device;

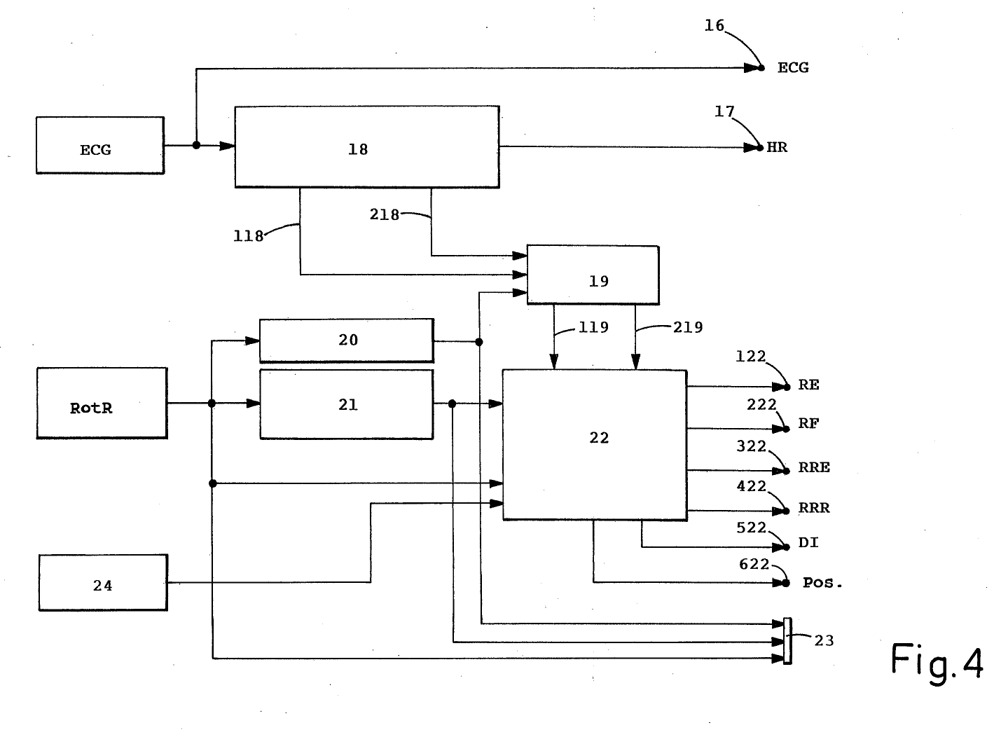

FIG. 4 is a block diagram of the system for collecting and processing data using the device according to the invention.

As shown in FIG. 1, in a preferred embodiment the device according to the invention comprises a casing 1 which is to be placed under the skin C of the patient's body, and which is therefore made from a material with a biocompatible outer surface, such as polyurethane. The casing 1 is surrounded by the coil 2 of a system for receiving electrical energy by inductive coupling and for transmitting the detected data to the outside, while the casing 1 also houses an internal unit 3 containing the integrated circuits with the necessary hardware and software for data acquisition and transmission, and with a posture sensor 9, such as a three-dimensional accelerometer, sensitive to gravitational acceleration (see below).

The casing 1 also houses one of the electrodes E2 for detecting the electrocardiographic signal from the heart of the patient in whom the device in question is to be implanted.

A catheter 4 runs from the casing 1 and is implanted in the patient's cardiovascular system, with its distal end 104 placed as closely as possible to the apex of the heart H, next to the left ventricle, for example by insertion through the great cardiac vein VM. In an intermediate part of the catheter 4, for example, there is at least one other electrode E1 which, together with the aforesaid electrode E2, enables the electrocardiographic signal to be detected by the known ECG procedure (see below). At the distal end 104 of the catheter 4 there is a sensor 5 for detecting the broadband signal of mechanical rotation rate, which is converted by the sensor to an electrical signal which, when processed by an operation of integration, supplies a signal relating to the rotation of the heart and, by an operation of derivation, supplies a signal relating to the mechanical vibrations corresponding to the heart sounds. Said sensor 5 can be composed, for example, of a miniature gyroscopic piezoelectric fork sensor, with a sensitivity range of approximately 0-1000 Hz, although it should be appreciated that other types of sensor can be used, provided that they are suitable for the purpose. For example, rotation sensors, rather than rotation rate sensors, can be used to obtain similar information simply by means of operations of derivation. In this case, the second derivative would correspond to the mechanical vibrations corresponding to the heart sounds as evaluated in the case described.

The implanted device can interact with an external programming and interrogation system, comprising a reception and transmission antenna 102 connected to a portable data collection unit 6, which can be provided with an alarm signalling device for responding to the detection of any anomalous data, and which, by means of any suitable data transfer means 7, can be connected by a wire or wireless link to an external server 8 provided with means for data acquisition, for the analysis of the data, and for commands and feedback signals to the patient.

FIG. 2 shows the following intercorrelated signals detected during experiments on animals. The first signal, at the top of the figure, corresponds to an electrocardiogram or ECG, in which the R, S and T waves of a cardiac cycle are marked. The new sensor 5 used in the device according to the invention, which as stated has a sensitivity in the range from 0 to 1000 Hz, intrinsically generates a broadband signal which corresponds to the second signal RotR shown in FIG. 2. By an operation of derivation, this signal RotR is processed to obtain the third signal d(RotR)/dt which contains the information relating to the mechanical vibrations corresponding on the heart sounds. The fourth signal, ∫RotRdt, corresponds to the angle of rotation of the heart and is obtained by an operation of integrating said signal RotR.

FIG. 2 also shows:

The vibrations V1 and V2 corresponding to the FHS and SHS respectively;

The end line A corresponding to the end of the vibrations V1, i.e. the “End of FHS”;

The end line B corresponding to the start of the vibrations V2, i.e. the “Onset of SHS”.

FIG. 2 also shows an example of combination of the said processed signals d(RotR)/dt and ∫RotRdt to characterize cardiac rotation with respect to systolic and diastolic phases, e.g. to identify:

MaxRotR (the peak of rotation rate during ejection);

MinRotR (the peak of rotation rate during relaxation/refilling);

The peak P1 of rotation during ejection;

The peak P2 of rotation during ventricular relaxation.

The automatic detection of the peak MaxRotR of the rotation rate of the cardiac apex, without reference to the mechanical phases of the heart cycle, is not reliable if measured as the absolute peak of the rotation rate signal, since this signal may show more than one peak. The peak of the rotation rate corresponding to the ejection phase is that which is found within a time interval between the first heart sound FHS and the second heart sound SHS, which can be detected from the d(RotR)/dt signal. The zero value of the rotation rate is found at the end of the said FHS, when the left ventricle is ready to start the blood ejection phase. This is because, at this instant, the heart has initiated the isovolumic contraction phase, making it possible to find the objective and reproducible zero point of each signal detected. In the absence of this important information, even the measurements of the positive and negative peaks would be affected by errors due to arbitrary assumptions relating to the zero reference point. This is the most well-defined signal and is essential for the precise and reproducible measurement of the peaks of the rotation rate and the peak of the rotation itself. As further example of combination of the said processed signals, by using the mechanical heart vibration signal d(RotR)/dt and the heart rotation signal ∫RotRdt it is possible to identify and signal correctly any dyssynchrony intervals (DI), corresponding to the time interval between the peak of said rotation signal and the closing of the aortic valve, identified by the mechanical vibrations corresponding to the SHS.

As shown in FIG. 3, the electrodes E1 and E2 of FIG. 1, used to acquire the patient's cardiac ECG, the sensor 5 for measuring the rotation and the mechanical vibrations corresponding to the heart sounds, and the three-dimensional sensor 9 for detecting the “Pos.” data relating to the patient's posture are connected via corresponding amplifiers 10, 110, 210 to a multiplexer 11, which in turn is connected to an A/D converter 12, which samples the signals from the three different channels at an appropriate frequency, for example at a sampling frequency of about 1 kHz. The amplifier 110 transmits the broadband signal of the rotation rate of the heat to the multiplexer 11. The data from the multiplexer 11 are sent to an analogue/digital converter 12, and from there to a RAM 13 and to a control unit 14 which acquires and processes the data and which operates in a feedback circuit with the units 11, 12 and 13. Clearly, the scope of the invention also includes the variant embodiment in which the analogue signals from the three different channels E1-E2, 5 and 9 are digitized by suitable converters before being sent to a multiplexer 11, which in this case would be of a completely digital type. By means of the telemetric unit 15, the units 13 and 14 can interact with the outside via the antenna 2 of the device, as shown in FIG. 1.

In the case of an implantable device which can also provide real-time treatment, the information detected by the system which has been described can be transmitted externally to other more complex processing systems, or can be processed internally and automatically for the purpose of therapeutic decision-making.

The block diagram in FIG. 4 summarizes the system for processing the data detected, amplified and transmitted by the system of FIG. 3 described above. The ECG is made directly available via the output 16 for the morphological analysis and analysis of the heart rhythm. The unit 18 supplies the heart rate HR to the output 17. The unit 18 identifies the R and T waves from the ECG. The outputs 118 and 218 of the unit 18 transfer the R wave signal and the T wave signal, respectively, to the processing unit 19, to enable the unit 19 to detect the first heart sound FHS and the second heart sound SHS.

The broadband heart signal RotR is sent to the units 20 and 21, which supply the analogue signal d(RotR)/dt and the analogue rotation signal ∫RotRdt respectively. The first unit 20 is connected to the unit 19 in such a way that its outputs 119, 219 can identify the first heart sound FHS and the second heart sound SHS, which, together with the signal from the unit 21 and the signal RotR, are sent to the unit 22, which processes the various data for the evaluation of the parameters relating to the rotation of the heart, and which supplies the measurement of the rotation RE relating to the ejection phase of the heart chamber at the output 122, supplies the measurement of the rotation RF relating to the refilling of the heart chamber at the output 222, supplies the measurement of the peak rotation rate RRE relating to the ejection phase at the output 322, supplies the measurement of the peak rotation rate RRR relating to the phase of relaxation and refilling of the heart chamber at the output 422, supplies the measurement of DI, in other words any dyssynchrony interval as described above with reference to FIG. 2, at the output 522, supplies the measurement of any other meaningful parameter derived from basic signal processing operations (i.e. sum, subtraction, multiplication, division, mean, calculation of the area underneath curves) and finally supplies the parameters relating to the patient's posture, obtained from the unit 24, at the output 622.

The analogue signals RotR, d(RotR)/dt and ∫RotRdt can be collected from the output 23.

There are evident advantages that can be obtained by the use of a single sensor 5 for supplying a signal relating to the rotation of the heart and to the heart sounds, which is not affected by external electrical signals, which can easily be processed without any need for synchronization with other physiological signals, and which is a source for the determination, by simple processing operations, of the aforesaid functions ∫RotRdt, d(RotR)/dt and for the estimation of any meaningful parameter (e.g. Dyssynchrony Interval DI) derived from the combination of said functions and from the application of simple signal processing operations to them. Clearly, the scope of the invention includes any solution other than that described which is capable of achieving the same ends, and therefore said sensor 5 can be replaced by or associated with one or more suitable motion sensors, such as accelerometers, capable of additionally detecting the translational movements of the heart in the radial and longitudinal directions, or other sensors of the gyroscopic type capable of detecting the rotation of the heart wall at a number of levels along the longitudinal axis of the heart, for example between the base and the intermediate fibres, or other devices including passive devices such as small permanent magnets which are suitably positioned and fixed on the heart walls and which therefore move with the heart walls and whose positions and movements can be detected by an external magnetic field sensors which can supply the information relating to both rotational and translational movements of the heart.

1. Implantable telemetric device for measuring electromechanical parameters of the heart, characterized in that it comprises a sensor (5) and corresponding processing means for detecting data relating to both the rotation of the heart and the mechanical vibrations which correspond to the first heart sound (FHS) and the second heart sound (SHS), and in that it comprises means which use these data for diagnostic and/or therapeutic and/or monitoring purposes.

2. Device according to claim 1, in which said sensor (5) is of the type which can generate a broadband electrical signal proportional to the rotation rate (RotR) of the heart and proportional to said mechanical vibrations corresponding to the heart sounds.

3. Device according to claim 2, characterized in that it comprises means which process the broadband signal generated by said sensor (5), by means of an operation of integration to supply a signal of the angle of rotation of the heart (∫RotRdt) and by means of an operation of derivation to obtain the signal [d(RotR)/dt] of the mechanical vibrations corresponding to the heart sounds (FHS, SHS).

4. Device according to claim 3, characterized in that it comprises means for detecting, on the basis of the end (A) of the first heart sound (End of FHS), the zero reference level of the signal (RotR) produced by said sensor (5) and of the integration corresponding to the signal (∫RotRdt) of the rotation of the heart, in order to enable the signal produced by said sensor (5) to be processed correctly.

5. Device according to claim 4, characterized in that it comprises means for detecting data derived from combination of said signals ∫RotRdt and d(RotR)/dt, such as data relating to any time interval (DI) that may be present between the peak (P2) of said heart rotation signal (∫RotRdt) and the second heart sound (SHS) obtained from the mechanical vibration signal, and data derived from basic signal processing operations applied to all said signals and in that it comprises means for using said data for diagnostic and/or therapeutic and/or monitoring purposes.

6. Device according to claim 1, characterized in that said sensor (5) can be associated with suitable means for acquiring the cardiac ECG of the patient detected by means of suitable electrodes (E1, E2) forming part of the implantable device.

7. Device according to claim 1, characterized in that said sensor (5) can be associated with suitable means for detecting the patient's posture, such as a three-dimensional accelerometer (9) which is sensitive to gravity, so as to associate the detected data with the corresponding and contingent posture of the patient.

8. Device according to claim 1, in which said sensor (5) is composed of a single sensor, for example a miniature gyroscopic sensor of the piezoelectric fork type or a rotation sensor of another type, provided that it is suitable for the purpose.

9. Device according to claim 1, in which said sensor (5) can be composed of or associated with one or more suitable motion sensors, such as accelerometers, capable of additionally detecting the translational movements of the heart in the radial and longitudinal directions, or other sensors of the gyroscopic type capable of detecting the rotation of the heart wall at a number of levels along the longitudinal axis of the heart, or other devices, including magnetic devices, whose movement can be detected by an external sensor which can supply the information relating to both the rotational and the translational movements of the heart.

10. Device according to claim 1, characterized in that it comprises at least one said sensor (5) for detecting the rotation and the mechanical vibrations of the heart corresponding to the heart sounds, with a corresponding amplifier (110), said amplifier being connected to a multiplexer (11), to an analogue/digital converter (12), to a RAM (13) and to a control unit (14) which acquires and processes the data and which operates in a feedback circuit with the upstream units (11, 12 and 13), provision being made to enable said memory unit (13) and control unit (14) to interact with the outside by means of a telemetric unit (15), using an antenna (2).

11. Device according to claim 10, characterized in that said multiplexer (11) is also connected to a means for acquiring the patient's cardiac ECG, with a corresponding signal amplifier (10) and a three-dimensional accelerometer (9) with a corresponding amplifier (210) which supplies the spatial coordinates relating to the patient's posture during the processing of the data.

12. Device according to claim 1, characterized in that it comprises a unit (18) which detects the R wave and T wave from the electrocardiographic signal (ECG), the outputs (118, 218) of this unit being enabled to transfer to a subsequent processing unit (19) the marker for the R wave and the marker for the T wave of the ECG respectively, provision being made to connect the broadband heart rotation rate signal (RotR) to the units (20, 21) which, respectively, supply the derived signal [d(RotR)/dt)] relating to the mechanical vibrations of the heart and the integrated signal (∫RotRdt) relating to the angle of rotation of the heart, the first of these units (20) being connected to said processing unit (19) whose outputs (119, 219) supply, respectively, the signal relating to the end of the first heart sound (End of FHS) and the signal relating to the start of the second heart sound (Onset of SHS), which, together with the signal received from said second unit (21) and the signal relating to the heart rotation rate (RotR), are sent to a final unit (22) which processes the various data for the measurement of the parameters relating to the heart rotation, and which has outputs (122, 222, 322, 422, 522, 622) supplying the measurement of rotation (RE) relating to the ejection phase of the heart chamber, the measurement of the rotation (RF) relating to the refilling phase of the heart chamber, the measurement (RRE) relating to the peak of the rotation rate during ejection, the measurement (RRR) relating to the peak of the rotation rate during the relaxation/refilling of the heart chamber, the measurement of any dyssynchrony interval (DI) that may be present, of any other meaningful parameter derived from basic signal processing operations and the parameters (Pos.) relating to the patient's posture.

13. Device according to claim 1, characterized in that it comprises a casing (1) which is to be placed under the patient's skin (C) and which is therefore covered with silicone or other suitable material, this casing being surrounded by the coil (2) of a system for receiving and transmitting power and data, and housing within it the unit (3) containing the integrated circuits with the necessary hardware and software for data acquisition and transmission and also housing a posture sensor (9) and one of the electrodes (E2) required for the detection of the electrocardiographic signal (ECG) from the patient's heart, said casing (1) being connected to a catheter (4) which is implanted in the patient's body and whose distal end (104) is placed as closely as possible to the apex of the patient's heart, on the left ventricle side, for example by insertion through the great cardiac vein (VM), said distal end housing said sensor (5) for detecting the electric signal relating to the rotation and to the mechanical vibrations of the heart during the systolic and diastolic phases, at least one other electrode (E1) being positioned in an intermediate part of the catheter (4) and being used in combination with the preceding electrode (E2) for detecting the electrocardiographic signal (ECG).

14. Device according to claim 13, characterized in that it can interact with an external programming and interrogation or dialogue system, comprising a reception and transmission antenna (102) connected to a portable data collection unit (6), which can be provided with an alarm signalling device for responding to the detection of any anomalous data, and which, by means of any suitable data transfer means (7), can be connected by a wire or wireless link to an external server (8) with means for data acquisition, for the processing and analysis of the data, and for the generation of commands and feedback signals to the patient.
